(19) United States
(12) Patent Application Publication
(10) Pub. No.: US 2004/0242887 A1
(43) Pub. Date: Dec. 2, 2004

(54) Deuterated n-substituted and alpha-substituted diphenylalkoxy acetic acid amino alkyl esters and medicaments containing these compounds

(76) Inventors: Rudolf-Giesbert Alken, Zepernick (DE); Johann Roither, Modling (AT); Wolf-Dietrich Hubner, Berlin (DE); Werner Hrachowina, Biedermannsdorf (AT); Thomas Stabingis, Berlin (DE)

Correspondence Address: Kriegsman & Kriegsman, 665 Franklin Street, Framingham, MA, 01702, US

(21) Appl. No.: 10/481,153

(22) PCT Filed: Jun. 17, 2002
(86) PCT No.: PCT/DE02/02260

(30) Foreign Application Priority Data
Jun. 17, 2001 (DE) 101 29 832.3

(51) Int. Cl. C07D 211/44; A61K 31/445; G01N 33/00

(52) U.S. Cl. 546/218; 514/317; 436/96

(57) ABSTRACT

The invention discloses deuterated N-substituted α,α-diphenyl α-alkoxy acetic acid aminoalkyl esters as well as the physiologically tolerated salts thereof. Furthermore, the invention concerns the use of deuterated N-substituted α,α-diphenyl α-alkoxy acetic acid aminoalkyl esters for the treatment of hypertonic functional states in the region of the urinary bladder as well as for the preparation of pharmaceutical drugs for the treatment of hypertonic functional states in the region of the urinary bladder.

In addition, the invention discloses pharmaceutical formulations containing deuterated N-substituted α,α-diphenyl α-alkoxy acetic acid aminoalkyl esters as well as the physiologically tolerated salts thereof for the treatment of hypertonic functional states in the region of the urinary bladder in addition to containing pharmaceutically tolerated adjuvants and/or additives.

The invention concerns deuterated N- and α-substituted diphenyl alkoxy acetic acid aminoalkyl esters as well as pharmaceutical drugs containing these compounds.

A known representative of the N- and α-substituted diphenyl alkoxy acetic acid aminoalkyl esters is propiverine (DD 106,643, DD 139,212, and DE 2,937,489). This compound is employed for the treatment of detrusor hyperactivity.

The problem of the present invention is to make available N- and α-substituted diphenyl alkoxy acetic acid aminoalkyl esters that, in comparison to the already known compounds, have improved pharmacokinetic and/or pharmacodynamic properties.

Surprisingly, it has now been found that the deuterated N- and α-substituted diphenyl alkoxy acetic acid aminoalkyl esters in accordance with the invention have appreciably better pharmacokinetic and/or pharmacodynamic properties than the non-deuterated compounds.

Thus, in accordance with the invention, the problem is solved by making available deuterated N- and α-substituted diphenyl alkoxy acetic acid aminoalkyl esters of the general formula I,

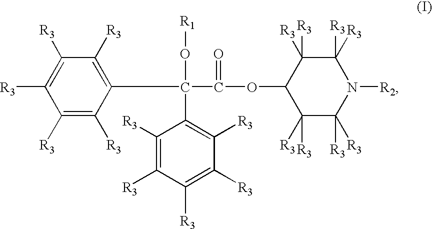

wherein

R₁ represents hydrogen, deuterium, an n-propyl group, or a singly deuterated, multiply deuterated, or perdeuterated n-propyl group,

R₂ is oxygen, a methyl group, or a mono-, di-, or trideuteromethyl group, and

R₃, independently of one another, indicates H or deuterium, wherein

at least one of the groups R₁, R₂, or R₃, independently of one another, is deuterium or contains deuterium,

as well as the physiologically tolerated salts thereof.

Especially preferred here are deuterated N- and α-substituted diphenyl alkoxy acetic acid aminoalkyl esters in accordance with the invention

wherein

R₁ represents an n-propyl group or a singly deuterated, multiply deuterated, or perdeuterated n-propyl group,

R₂ is a methyl group or a mono-, di-, or trideuteromethyl group, and

R₃, independently of one another, indicates H or deuterium, wherein

at least one of the groups R., R₂, or R₃, independently of one another, is deuterium or contains deuterium,

as well as the physiologically tolerated salts thereof.

Furthermore, especially preferred are deuterated N- and α-substituted diphenyl alkoxy acetic acid aminoalkyl esters in accordance with the invention

wherein

R₁ represents an n-propyl group or a singly deuterated, multiply deuterated, or perdeuterated n-propyl group,

R₂ is oxygen, and

R₃, independently of one another, indicates H or deuterium, wherein at least one of the groups R., R₂, or R₃, independently of one another, is deuterium or contains deuterium,

as well as the physiologically tolerated salts thereof.

Especially preferred in accordance with the invention are the following N- and α-substituted diphenyl alkoxy acetic acid aminoalkyl esters deuterated in accordance with the invention:

2,2-diphenyl-2-(d-hydroxy)acetic acid N-methyl-4-piperidinyl ester,

2,2,-diphenyl-2-hydroxyacetic acid N-trideteuromethyl-4-piperidinyl ester,

2,2-diphenyl-2-(d-hydroxy)acetic acid N-oxido-4-piperidinyl ester,

2,2-bis(pentadeuterophenyl)-2-(d-hydroxy)acetic acid N-trideuteromethyl-4-perdeuteropiperidinyl ester,

2,2-bis(pentadeuterophenyl)-2-hydroxyacetic acid N-trideuteromethyl-4-perdeuteropiperidinyl ester,

2,2-bis(pentadeuterophenyl)-2-(perdeuteropropyloxy)acetic acid N-oxido-4-perdeuteropiperidinyl ester,

2,2-diphenyl-2-(perdeuteropropyloxy)acetic acid N-methyl-4-piperidinyl ester,

2,2-diphenyl-2-(perdeuteropropyloxy)acetic acid N-trideuteromethyl-4-piperidinyl ester,

2,2-diphenyl-2-(perdeuteropropyloxy)acetic acid N-monodeuteromethyl-4-piperidinyl ester,

2,2-diphenyl-2-(perdeuteropropyloxy)acetic acid N-dideuteromethyl-4-piperidinyl ester,

2,2-diphenyl-2-(perdeuteropropyloxy)acetic acid N-oxido-4-piperidinyl ester,

2,2-diphenyl-2-(3, 3, 3-trideuteropropyloxy)acetic acid N-methyl-4-piperidinyl ester,

2,2-diphenyl-2-(3, 3, 3-trideuteropropyloxy)acetic acid N-trideuteromethyl-4-piperidinyl ester,

2,2-diphenyl-2-(3, 3, 3-trideuteropropyloxy)acetic acid N-monodeuteromethyl-4-piperidinyl ester,

2,2-diphenyl-2-(3, 3, 3-trideuteropropyloxy)acetic acid N-dideuteromethyl-4-piperidinyl ester,

2,2-diphenyl-2-(3, 3, 3-trideuteropropyloxy)acetic acid N-oxido-4-piperidinyl ester,

2,2-diphenyl-2-(perdeuteropropyloxy)acetic acid N-methyl-4-perdeuteropiperidinyl ester,

2,2-diphenyl-2-(perdeuteropropyloxy)acetic acid N-trideuteromethyl-4-perdeuteropiperidinyl ester,

2,2-diphenyl-2-(perdeuteropropyloxy)acetic acid N-monodeuteromethyl-4-perdeuteropiperidinyl ester,

2,2-diphenyl-2-(perdeuteropropyloxy)acetic acid N-dideuteromethyl-4-perdeuteropiperidinyl ester,

2,2-diphenyl-2-(perdeuteropropyloxy)acetic acid N-oxido-4-perdeuteropiperidinyl ester,

2,2-bis(pentadeuterophenyl)-2-(perdeuteropropyloxy)acetic acid N-trideuteromethyl-4-perdeuteropiperidinyl ester,

2,2-bis(pentadeuterophenyl)-2-(perdeuteropropyloxy)acetic acid N-oxido-4-perdeuteropiperidinyl ester,

2,2-bis(pentadeuterophenyl)-2-(perdeuteropropyloxy)acetic acid N-methyl-4-piperidinyl ester, and

2,2-bis(pentadeuterophenyl)-2-(perdeuteropropyloxy)acetic acid N-oxido-4-piperidinyl ester,

Preferred is the use of the deuterated N- and α-substituted diphenyl alkoxy acetic acid aminoalkyl esters in accordance with the invention as well as the physiologically tolerated salts thereof for the treatment of hypertonic functional states in the region of the urinary bladder.

Especially preferred is the use of the deuterated N- and α-substituted diphenyl alkoxy acetic acid aminoalkyl esters in accordance with the invention as well as the physiologically tolerated salts thereof for the preparation of pharmaceutical drugs for the treatment of hypertonic functional states in the region of the urinary bladder.

Especially preferred are pharmaceutical formulations that contain the deuterated N- and α-substituted diphenyl alkoxy acetic acid aminoalkyl esters in accordance with the invention as well as the physiologically tolerated salts thereof for the treatment of hypertonic functional states of the urinary bladder in addition to containing pharmaceutically tolerated adjuvants and/or additives.

Another subject of the present invention is comprised of pharmaceutical formulations for the percutaneous and/or transdermal application of the deuterated N- and α-substituted diphenyl alkoxy acetic acid aminoalkyl esters in accordance with the invention as well as the physiologically tolerated salts thereof.

The preparation of the N- and α-substituted diphenyl alkoxy acetic acid aminoalkyl esters in accordance with the invention is in itself known and can take place as described in DD 106,643.

To this end, the methyl ester of benzilic acid is transesterified with an N-substituted amino alcohol in the presence of a catalytically acting, strong base with simultaneous azeotropic removal of methanol and converted into the α-chloro compound by reaction with thionyl chloride. The halogen compound is brought to reaction with an alcohol, water, or D₂O and affords, after a reaction time of up to 10 hours, the desired N- and α-substituted diphenyl alkoxy acetic acid amino alkyl ester in the form of its acid chloride.

The preparation of the deuterated compounds thus takes place by reaction of the corresponding deuterated starting materials, such as d-benzilic acid methyl ester or deuterated N-substituted amino alcohols, or, as described in Embodiment Example 1, by reaction of the α-chloro compound with deuterated alcohol.

Conventional physiologically tolerated inorganic and organic acids are, for example, hydrochloric acid, hydrobromic acid, phosphoric acid, sulfuric acid, oxalic acid, maleic acid, fumaric acid, lactic acid, tartaric acid, malic acid, citric acid, salicylic acid, adipic acid, and benzoic acid. Further salts that can be used are described, for example, in Fortschritte der Arzneimittelforschung [Progress in Drug Research], Vol. 10, pages 224-225, Birkhäuser Publishing Co., Basel and Stuttgart, 1966, and Journal of Pharmaceutical Sciences, Vol. 66, pages 1-5 (1977).

The acid addition salts are obtained, as a rule, in a way that is in itself known by mixing the free base or solutions thereof with the corresponding acid or solutions thereof in an organic solvent, such as, for example, in a lower alcohol, like methanol, ethanol, n-propanol, or isopropanol, or in a lower ketone, like acetone, methyl ethyl ketone, or methyl isobutyl ketone, or in an ether, like diethyl ether, tetrahydrofuran, or dioxane. In order to achieve better separation of the crystals, it is also possible to use mixtures of the solvents mentioned. Beyond this, it is possible to prepare physiologically tolerated aqueous solutions of acid addition salts of the compounds used in accordance with the invention in an aqueous acid solution.

The acid addition salts of the compounds in accordance with the invention can be transformed into the free base in ways that are in themselves known—for example, with alkalies or ion exchangers. Further salts can be obtained from the free base by reaction with inorganic or organic acids, in particular with those suitable for the formation of salts that can be used therapeutically. These or else other salts of the new compound, such as, for example, the picrate, can also serve for the purification of the free base by transforming the free base into a salt, separating the latter, and liberating the base once again from the salt.

The subject of the present invention is also comprised of pharmaceutical drugs for oral, rectal, subcutaneous, intravenous, or intramuscular application that, in addition to conventional carriers and diluents, contain a compound of the general formula I or its acid addition salt as the active ingredient.

The pharmaceutical drugs of the invention are prepared in a known way in a suitable dosage with the conventional solid or liquid carriers or diluents and the conventionally used technical pharmaceutical adjuvants depending on the desired kind of application. The preferred formulations consist in a form of administration that is suitable for oral application. Such forms of administration are, for example, tablets, film tablets, dragées, capsules, pills, powders, solutions, or suspensions or depot forms.

Obviously, parenteral formulations, such as injection solutions, also come into consideration. Furthermore, suppositories are also mentioned as formulations by way of example.

Corresponding tablets can, for example, be obtained by mixing the active ingredient with known adjuvants, such as, for example, inert diluents, like dextrose, sugar, sorbitol, mannitol, polyvinyl pyrrolidone, disintegrators, such as cornstarch or alginic acid, binders, such as starches or gelatins, lubricants, such as magnesium stearate or talc, and/or means for achieving a depot effect, such as carboxylpolymethylene, carboxymethylcellulose, cellulose acetate phthalate, or polyvinyl acetate. The tablets can also consist of several layers.

In a corresponding manner, dragées can be prepared by coating cores, prepared in analogy to the tablets, with substances usually used in dragée coats, such as, for example, polyvinyl pyrrolidone or shellac, gum arabic, talc, titanium dioxide, or sugar. Here, the dragée shell can also consist of several layers, wherein the adjuvants mentioned above for the tablets can be used.

Solutions or suspensions containing the active ingredient used in accordance with the invention can contain, in addition, substances that improve taste, such as saccharin, cyclamate, or sugar, as well as, for example, flavoring substances, such as vanilla or orange extract. In addition, they can contain suspending agents, such as sodium carboxymethylcellulose, or preservatives, such as p-hydroxybenzoates. For example, capsules that contain active ingredients can be prepared by mixing the active ingredient with an inert carrier, such as lactose or sorbitol, followed by encapsulation in gelatin capsules.

Suitable suppositories can be prepared, for example, by admixture with carriers, such as neutral fats or polyethylene glycol or their derivatives, that are provided for this purpose.

The preparation of the pharmaceutical drugs in accordance with the invention for percutaneous application is known to the person skilled in the art. In the preparation of the pharmaceutical drugs in accordance with the invention for transdermal application, the adjuvants and enhancers that are in themselves known are used.

The preparation of the pharmaceutical formulations in accordance with the invention is in itself known and is described in handbooks known to the person skilled in the art, such as, for example Hager's Handbuch [Hager's Handbook] (5th) 2, 622-1045; List et al., Arzneiformenlehre [Drug Forms], Stuttgart: Wiss. Publishing Co. 1985; Sucker et al., Pharmazeutische Technologie [Pharmaceutical Technology], Stuttgart: Thieme 1991; Ullmann's Enzyklopädie [Ullmann's Encyclopedia] (5th) A 19, 241-271; Voigt, Pharmazeutische Technologie [Pharmaceutical Technology], Berlin: Ullstein Mosby 1995.

The pharmaceutical drugs prepared in this way can be used for the treatment of hypertonic functional states in the region of the urinary bladder. Included in the symptoms here are involuntary discharge of urine (enuresis), pathologically frequent urination (urge incontinence), and painful urinary bladder cramps (tenesmus).

The compounds in accordance with the invention have a number of advantages over compounds known in the prior art, which do not bear any deuterium. The deuteration brings about a change in metabolism in the organism. In particular, the hydroxylation on the phenyl group is impeded, this leading to a reduced first-pass effect. In this way it is possible to change the dosage and to create longer-acting formulations, which, in the form of depot formulations, can also improve compliance.

In addition, the pharmacodynamics are also changed, because the deuterated compounds form completely different hydrate shells, so that the distribution in the organism differs markedly from that of the non-deuterated compounds.

It is possible in this way to develop completely new forms of formulation.

The following example illustrates the invention:

### Example 1

Preparation of the 1-methylpiperidyl 4-hydrochloride ester of α,α-diphenyl-d7-propyloxyacetic acid

24.2 g of the methyl benzilate, 0.05 g of sodium (dissolved in 3 mL of methanol), and 11.75 g of N-methyl-4-piperidinol are heated in a stirred solvent mixture consisting of 80 mL of toluene and 200 mL of benzene for 4 hours at 110° C. During this time, approximately 32 mL of methanol azeotrope are distilled off. Subsequently, an additional 65 to 70 mL of methanol azeotrope are distilled off and the separated solvent is [replaced] by pure toluene. Then, 0.1 mL of dimethylformamide is added and, after heating the solution to 100 to 105° C., 13.2 g of thionyl chloride are added under stirring within one hour. SO₂/HCl evolution commences and the reaction temperature drops to 90 to 85° C. Once the 1-methylpiperidyl 4-hydrochloride ester of α,α-diphenyl-α-chloroacetic acid begins to crystallize out, heating up to 110° C. is carried out carefully, as a function of the gas evolution, and excess thionyl chloride and solvent are removed as much as possible with an increasing water-jet vacuum. Under stirring, 15 mL of d9-n-propanol are added and the remaining toluene is distilled off azeotropically until the internal temperature has reached 100° C. The reaction batch is heated for 10 hours at reflux, this resulting in the occurrence of a temperature drop to approximately 93° C. After removal of 50 to 60 mL of hydrogen chloride and water-containing n-propanol, activated carbon is added and the solution is filtered while still hot. Subsequently, the solution is cooled and the product is filtered off, washed with a small amount of n-propanol, and dried.

Addition of n-hexane to the mother liquor affords additional product, which is recrystallized with a small amount of activated carbon from a small amount of n-propanol.

Yield: 34.52 g; 84%

Melting point: 212-217° C.

Calculated

| | | | | | |
|---|---|---|---|---|---|
| C: | 67.22%, | H: | 9.07%, | N: | 3.41% |

Found

| | | | | | |
|---|---|---|---|---|---|
| C: | 67.24%, | H: | 9.04%, | N: | 3.42% |

1. Deuterated N- and α-substituted diphenyl alkoxy acetic acid aminoalkyl esters of the general formula I, 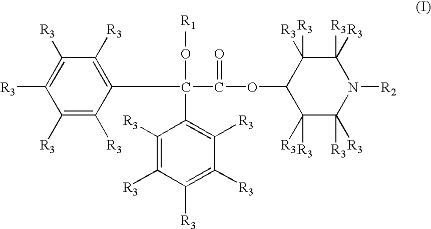
wherein
R₁ represents hydrogen, deuterium, an n-propyl group, or a singly deuterated, multiply deuterated, or perdeuterated n-propyl group,
R₂ is oxygen, a methyl group, or a mono-, di-, or trideuteromethyl group, and
R₃, independently of one another, indicates h or deuterium, wherein at least one of the groups R₁, R₂, or R₃, independently of one another, is deuterium or contains deuterium, as well as the physiologically tolerated salts thereof.

2. The deuterated N- and α-substituted diphenyl alkoxy acetic acid aminoalkyl esters of the general formula I according to claim 1
wherein
R₁ represents an n-propyl group or a singly deuterated, multiply deuterated, or perdeuterated n-propyl group,
R₂ is a methyl group or a mono-, di-, or trideuteromethyl group, and
R₃, independently of one another, indicates H or deuterium, wherein at least one of the groups R₁, R₂, or R₃, independently of one another, is deuterium or contains deuterium,
as well as the physiologically tolerated salts thereof.

3. The deuterated N- and α-substituted diphenyl alkoxy acetic acid aminoalkyl esters of the general formula I according to claim 1
[wherein]
R₂ represents an n-propyl group or a singly deuterated, multiply deuterated, or perdeuterated n-propyl group,
R₂ is oxygen, [and]
R₃, independently of one another, indicates H or deuterium, wherein at least one of the groups R₁, R₂, or R₃, independently of one another, is deuterium or contains deuterium,
as well as the physiologically tolerated salts thereof.

4. The deuterated N- and α-substituted diphenyl alkoxy acetic acid aminoalkyl esters according to claim 1, namely:
,2-diphenyl-2-(d-hydroxy)acetic acid N-methyl-4-piperidinyl ester,
2,2,-diphenyl-2-hydroxyacetic acid N-trideteuromethyl-4-piperidinyl ester,
2,2-diphenyl-2-(d-hydroxy)acetic acid N-oxido-4-piperidinyl ester,
2,2-bis(pentadeuterophenyl)-2-(d-hydroxy)acetic acid N-trideuteromethyl-4-perdeuteropiperidinyl ester,
2,2-bis(pentadeuterophenyl)-2-hydroxyacetic acid N-trideuteromethyl-4-perdeuteropiperidinyl ester,
2,2-bis(pentadeuterophenyl)-2-(perdeuteropropyloxy)acetic acid N-oxido-4-perdeuteropiperidinyl ester,
2,2-diphenyl-2-(perdeuteropropyloxy)acetic acid N-methyl-4-piperidinyl ester,
2,2-diphenyl-2-(perdeuteropropyloxy)acetic acid N-trideuteromethyl-4-piperidinyl ester,
2,2-diphenyl-2-(perdeuteropropyloxy)acetic acid N-monodeuteromethyl-4-piperidinyl ester,
2,2-diphenyl-2-(perdeuteropropyloxy)acetic acid N-dideuteromethyl-4-piperidinyl ester,
2,2-diphenyl-2-(perdeuteropropyloxy)acetic acid N-oxido-4-piperidinyl ester,
2,2-diphenyl-2-(3, 3, 3-trideuteropropyloxy)acetic acid N-methyl-4-piperidinyl ester,
2,2-diphenyl-2-(3,3,3-trideuteropropyloxy)acetic acid N-trideuteromethyl-4-piperidinyl ester,
2,2-diphenyl-2-(3,3,3-trideuteropropyloxy)acetic acid N-monodeuteromethyl-4-piperidinyl ester,
2,2-diphenyl-2-(3,3,3-trideuteropropyloxy)acetic acid N-dideuteromethyl-4-piperidinyl ester,
2,2-diphenyl-2-(3, 3, 3-trideuteropropyloxy)acetic acid N-oxido-4-piperidinyl ester,
2,2-diphenyl-2-(perdeuteropropyloxy)acetic acid N-methyl-4-perdeuteropiperidinyl ester,
2,2-diphenyl-2-(perdeuteropropyloxy)acetic acid N-trideuteromethyl-4-perdeuteropiperidinyl ester,
2,2-diphenyl-2-(perdeuteropropyloxy)acetic acid N-monodeuteromethyl-4-perdeuteropiperidinyl ester,
2,2-diphenyl-2-(perdeuteropropyloxy)acetic acid N-dideuteromethyl-4-perdeuteropiperidinyl ester,
2,2-diphenyl-2-(perdeuteropropyloxy)acetic acid N-oxido-4-perdeuteropiperidinyl ester,
2,2-bis(pentadeuterophenyl)-2-(perdeuteropropyloxy)acetic acid N-trideuteromethyl-4-perdeuteropiperidinyl ester,
2,2-bis(pentadeuterophenyl)-2-(perdeuteropropyloxy)acetic acid N-oxido-4-perdeuteropiperidinyl ester,
2,2-bis(pentadeuterophenyl)-2-(perdeuteropropyloxy)acetic acid N-methyl-4-piperidinyl ester, and
2,2-bis(pentadeuterophenyl)-2-(perdeuteropropyloxy)acetic acid N-oxido-4-piperidinyl ester.

5. The use of the deuterated N- and α-substituted diphenyl alkoxy acetic acid aminoalkyl esters according to one of the claims 1 to 4 as well as of the physiologically tolerated salts thereof for the treatment of hypertonic functional states in the region of the urinary bladder.

6. The use of the deuterated N- and α-substituted diphenyl alkoxy acetic acid aminoalkyl esters according to one of the claims 1 to 4 as well as of the physiologically tolerated salts thereof for the preparation of pharmaceutical drugs for the treatment of hypertonic functional states in the region of the urinary bladder.

7. A pharmaceutical formulation containing N- and α-substituted diphenyl alkoxy acetic acid aminoalkyl esters according to one of the claims 1 to 4 as well as of the physiologically tolerated salts thereof for the preparation of pharmaceutical drugs for the treatment of hypertonic functional states of the urinary bladder in addition to containing pharmaceutically tolerated adjuvants and/or additives.

8. A pharmaceutical formulation for the percutaneous and/or transdermal application of deuterated N- and α-substituted diphenyl alkoxy acetic acid aminoalkyl esters according to one of the claims 1 to 4 as well as of the physiologically tolerated salts thereof.
